(19) United States
(12) Patent Application Publication
(10) Pub. No.: US 2012/0172632 A1
(43) Pub. Date: Jul. 5, 2012

(54) CONTINUOUS METHOD FOR THE HYDROGENATION OF AROMATIC COMPOUNDS

(75) Inventors: Sabrina Marie, Revest-Les-Roches (FR); Alexandre Trani, Carros (FR); Raimondo Pilia, Cagnes sur Mer (FR)

(73) Assignee: AET Group, Cagnes sur Mer (FR)

(21) Appl. No.: 13/381,999

(22) PCT Filed: Jun. 10, 2010
(86) PCT No.: PCT/FR2010/000425

§ 371 Date: Feb. 2, 2012

(30) Foreign Application Priority Data
Jul. 3, 2009 (FR) 09/03266

(51) Int. Cl. C07C 29/20

(52) U.S. Cl. 568/832

(57) ABSTRACT

The invention relates to a continuous method for the catalytic hydrogenation of an aromatic compound into a cycloaliphatic compound, wherein said method is carried out in a piston reactor provided with a mechanical axially agitating means and comprises continuously feeding a liquid phase comprising said aromatic compound and a catalyst dispersed to the liquid phase, subjecting said liquid phase, at a temperature of between 100° C. and 300° C. and while being mechanical axially agitated, to the effects of a hydrogen pressure of between 10 and 250 bars in the presence of said catalyst dispersed in the liquid phase for a residence time of between 1 second and 10 minutes, and removing the liquid phase from the reactor.

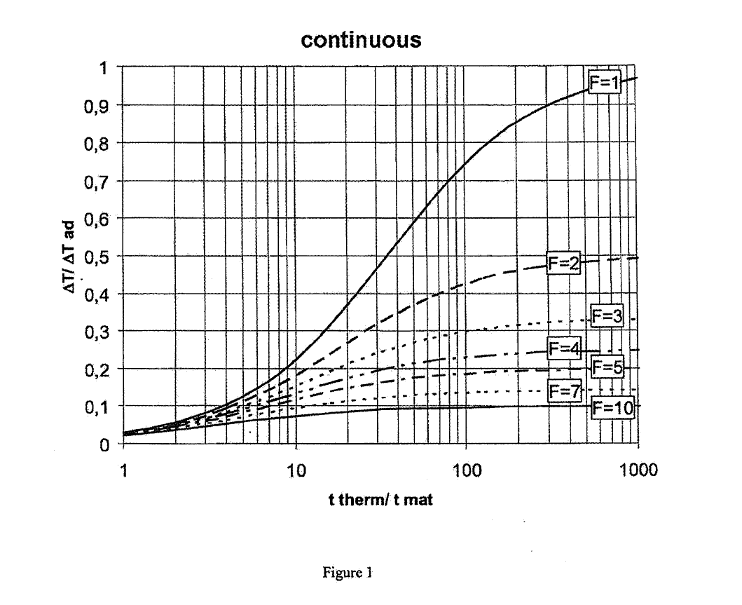

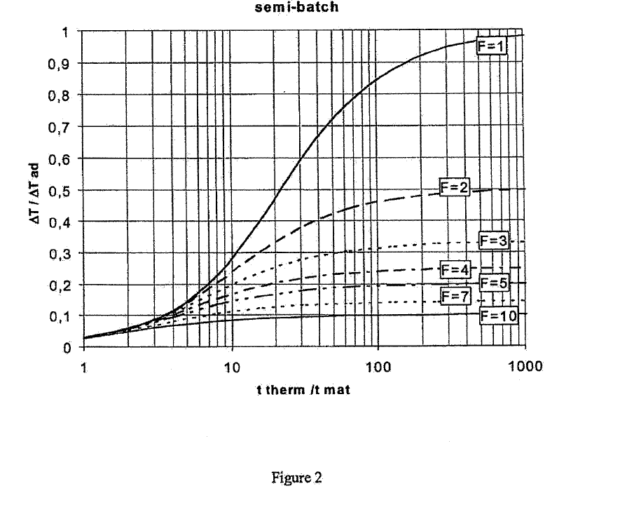

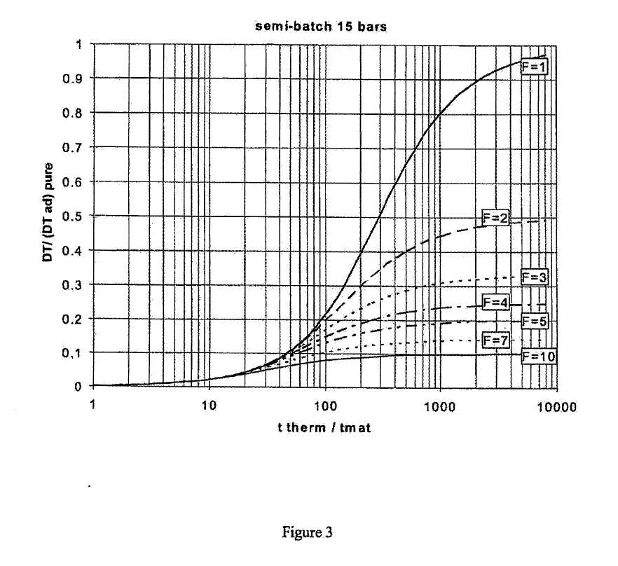

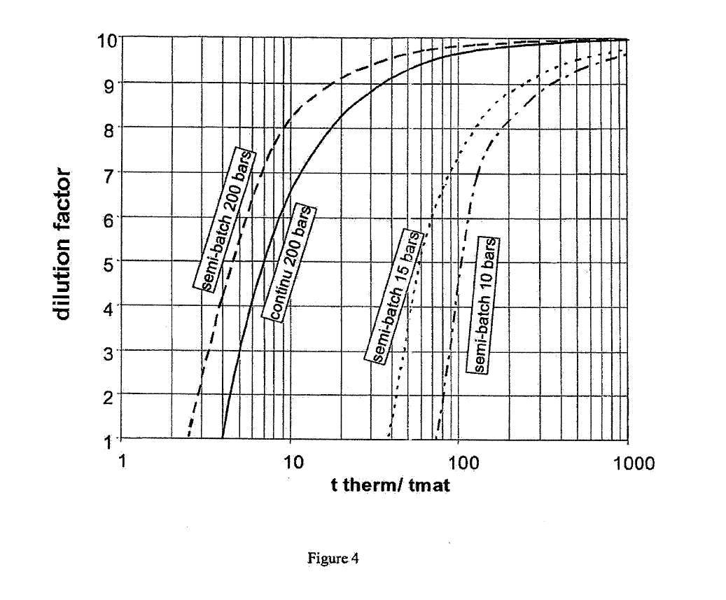

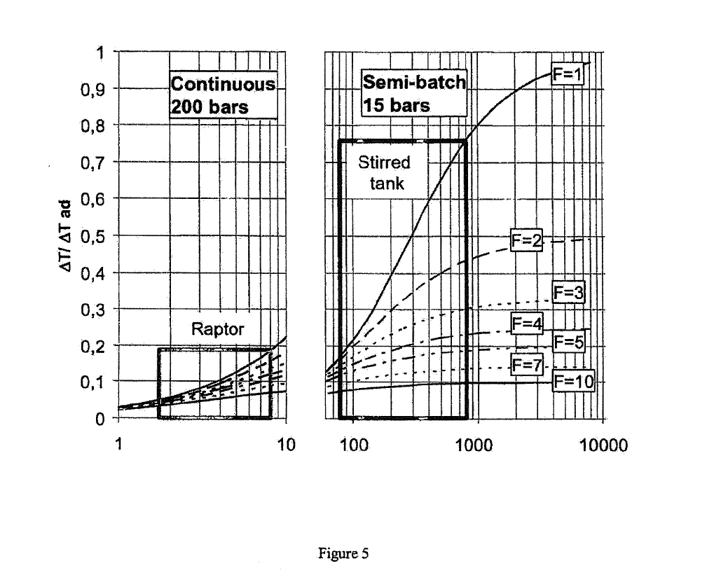

### CROSS-REFERENCE TO RELATED APPLICATIONS

The present application is a National Stage Application of PCT International Application No. PCT/FR2010/000425 (filed on Jun. 10, 2010), under 35 U.S.C. §371, which claims priority to French Patent Application No. 0903266 (filed on Jul. 3, 2009), which are each hereby incorporated by reference in their respective entireties.

### FIELD OF THE INVENTION

This invention relates to a continuous method for the catalytic hydrogenation of aromatic compounds, and in particular substituted phenols. This method can be used in fine chemistry for the industrial preparation of cyclohexane derivatives.

### BACKGROUND OF THE INVENTION

Cyclohexane derivatives contain a structural element, the cyclohexane ring, which is part of numerous molecules used, often in relatively small amounts, as commercial products or active substances in numerous sectors of the industry, such as in the pharmaceutical, food, cosmetics or photographic industries. The cyclohexane ring is relatively difficult to synthesize by cyclization of aliphatic chains. Alternatively, it can be obtained by a Diels-Alder-type cycloaddition. It can also be obtained by hydrogenation of benzene derivatives. Indeed, benzene derivatives are relatively easy to obtain, including in industrial quantities, as certain derivatives of benzene are part of the fractions obtained from oil distillation. However, the hydrogenation of benzene derivatives often leads to a mixture of isomers. As an example, it is well known (see, for example: Streitwieser/Heathcock, “Introduction to Organic Chemistry”, McMillan 1976, page 623) that the hydrogenation of 4-methylphenol in the presence of Ni and at a temperature of 150° C. produces a mixture of cis-4-methylcyclohexanol and trans-4-methylcyclohexanol.

Numerous methods are known for hydrogenating benzene and benzene derivatives. In general, they are methods conducted under high hydrogen pressure and at high temperature, which are carried out in the presence of a catalyst. The reaction is exothermic. A large number of published works relate to the improvement of catalysts.

Some of the prior art methods are carried out in “batch” mode, i.e. in a static agitated autoclave reactor, in which the starting compound is placed in contact with the pressurized hydrogen and at a temperature higher than ambient temperature for a long enough time for the reaction to be complete. The hydrogen can be applied statically or in the form of a flow passing through the reactor. At the end of the reaction, the reaction products are recovered.

As an example, the French patent application FR 2 398 709 (ANIC S.p.a.) describes a method for hydrogenation of aromatic compounds in the presence of a fixed bed catalyst, performed in an autoclave at a temperature of between 100° C. and 200° C., and at a pressure of between 1 and 150 bars. U.S. Pat. No. 2,857,432 (Allied Chemical Corporation) describes a method for hydrogenation of phenol into cyclohexanone in the presence of a finely divided palladium catalyst in a continuous reactor at a temperature of between 100° C. and 200° C.; the document teaches that, at high pressure, secondary products form.

The French patent application FR 2 217 293 (Diamond Shamrock Corp.) describes a method for hydrogenation of benzene compounds performed in an autoclave, in the presence of a catalyst in the form of a paste or a filtration cake, at a pressure of between 10 and 100 bar and at a temperature of between 70° C. and 200° C.

U.S. Pat. No. 6,489,520 (Haarmann & Reimer GmbH) describes a method for selective hydrogenation of 2-t-butylphenol into cis-t-butylcyclohexanol in the presence of a dispersed catalyst, at a temperature of between 50° C. and 200° C., and preferably between 90° C. and 130° C., and at a pressure of between 1 and 100 bar, and preferably between 10 and 20 bar. This reaction is very long, and lasts between 2 and 100 hours.

EP Patent Application 0 703 210 A1 (Fuji Photo Film Co., Ltd.) describes a method for hydrogenation in the presence of a dispersed catalyst, suitable for the preparation of numerous substituted cyclohexanols from corresponding phenol compounds. This method is performed in an autoclave in the presence of a solvent or without solvent, at a temperature of between 50° C. and 300° C., preferably between 60° C. and 250° C., and at a pressure of between 15 and 250 bar, preferably between 15 bar and 250 bar.

EP Patent Application 0 427 965 (Firmenich & Cie) describes a continuous method for hydrogenation of 4-t-butyl-phenol into 4-t-butyl-cyclohexanol in the presence of a catalytic system consisting of rhodium on a substrate in combination with HBF₄ or one of its organic derivatives, in a very specific proportion, at a preferred temperature of 40° C. to 130° C., in the presence of a solvent, in an autoclave reactor at a pressure of between 3 and 200 bar.

Continuous methods are also known in which a continuous flow of starting compounds is treated with hydrogen, and said starting compounds enter the reactor, pass through the reactor during their hydrogenation, and leave the reactor, all in a continuous flow.

U.S. Pat. No. 5,874,648 and U.S. Pat. No. 6,031,140 (Bayer AG) describe a method for hydrogenation of an isocamphylguaiacol, which is a compound including a benzene ring substituted by a an —OH group (and possibly an —OCH₃ group in the ortho position with respect to the —OH group) and by an isocamphyl group, into isocamphylcyclohexanol. This reaction can be performed in the liquids phase, in a single step, with or without a solvent, at a temperature of between 140° C. and 280° C., and preferably between 180° C. and 250° C., at a hydrogen pressure of between 50 bar and 400 bar, and preferably between 150 bar and 300 bar, in the presence of a fixed bed catalyst.

U.S. Pat. No. 5,942,645 (BASF AG) describes a method for hydrogenation of compounds including a benzene ring substituted by an —OH group and possibly by a C1 to C10 alkyl radical, substituted or not, and/or by an alkoxy radical, into the corresponding cycloaliphatic compounds. This method is performed in the presence of a fixed bed catalyst including at least ruthenium, and this catalyst is deposited on a porous substrate. The reaction can be conducted without solvent, but the presence of a solvent is preferred. The hydrogen pressure is at least 50 bar and preferably between 150 bar and 300 bar. The temperature is between 100° C. and 270° C., and preferably between 150° C. and 220° C. The reaction can be conducted in continuous mode.

U.S. Pat. No. 5,189,233 (Texaco Chemical Company) describes a method for hydrogenation of benzene in which the benzene is exposed first to a first fixed bed catalyst with moderate activity, then the reaction mixture is exposed to a second fixed bed catalyst, more active than the first. The method is conducted at a temperature of between 40° C. and 300° C., preferably between 75° C. and 230° C., and at a pressure of between 35 bar and 275 bar, without solvent, in a tubular continuous flow reactor. This two-step hydrogenation approach performed in a continuous reactor, using two fixed bed catalysts that differ in their activity, is also found in U.S. Pat. No. 4,551,564 (Chemische Werke Hüls AG), which seeks to improve the yield of 2- and 4-t-butyl-cyclohexanol cis isomers.

French patent application FR 2 040 257 (Badische Anilin- & Soda-Fabrik AG) describes a method for hydrogenation of aromatic hydrocarbons into cycloaliphatic compounds, with or without a solvent, at a temperature of between 100° C. and 400° C., and preferably between 20 and 700 bar, and more specifically between 50 and 400 bar, in a tubular reactor, in continuous mode and in the presence of a fixed bed catalyst.

U.S. Pat. No. 3,700,742 (Universal Oil Products Co.) describes a continuous method for hydrogenation of aromatic hydrocarbon fractions, such as those resulting from oil distillation processes, in the presence of a fixed bed catalyst, at a pressure of between 35 bars and 140 bars and at a temperature of between 90° C. and 425° C. This document relates to a context different from that of fine chemistry, with the objective of obtaining pure fractions of well-defined products.

Batch-type reactors and continuous reactors differ significantly, for technical and practical reasons. Technically, for an exothermic reaction such as hydrogenation of aromatic compounds, the transfer of heat is a critical parameter, both for the design of the reactor and for the performance of the method. It is in particular for a batch-type reactor that the increase in the size of the reactor (“scaling up”) and therefore the increase in its production capacity, is limited by the heat transfer. The more the size of the reactor is increased to enhance productivity, the more the heat transfer is degraded. In high-capacity reactors, it is often necessary to dilute the reagents by adding solvent in order to be capable of controlling the exothermics of the reaction. However, the addition of a solvent then requires a separation step in order to isolate the reaction product from the solvent.

At the practical level, a discontinuous method requires, at the end of the reaction, the depressurization of the reactor, and the cooling, the opening and the emptying of same in order to recover the reaction mixture under good safety conditions; these discontinuous operations are complex and labor intensive. In addition, the investment cost of an autoclave-type reactor is high. Therefore, there is a tendency to prefer a continuous method, at least for products to be produced in a relatively large amount. The mass transfer is one of the general problems of chemical reactors, and hydrogenation reactors in particular. In discontinuous mode (“batch” mode), agitation systems have been developed specifically to improve the mass transfer. In continuous mode, without agitation, the mass transfer can be relatively ineffective.

In both types of methods, the use of solvents has disadvantages with regard to energy (cost of separation, heating of a larger mass), environment (waste) and cost (investment cost of the separation step, cost of the solvent, reduction in reaction rates).

### SUMMARY OF THE INVENTION

This invention therefore relates to a continuous method for preparing cyclohexane derivatives from benzene compounds, which is simple, easy to control, with high yields and selectivity, and which eliminates the need to add and subsequently separate solvent.

The invention relates to a continuous method for catalytic hydrogenation of an aromatic compound, preferably a mono- or poly-substituted derivative of benzene, into a cycloaliphatic compound, in which the method is performed in a piston reactor, preferably having a cylindrical shape, and the reactor is equipped with axial mechanical agitation means, and in which: at least one liquid phase that includes the aromatic compound and a catalyst dispersed in the liquid phase is continuously supplied, preferably at one end of the reactor, the liquid phase being subjected, at a temperature of between 100° C. and 300° C. and under axial mechanical agitation, to the influence of a hydrogen pressure of between 10 and 250 bar, and preferably between 50 and 250 bar, in the presence of the catalyst dispersed in the liquid phase, for a passage time “t” of between 10 seconds and 10 minutes, preferably between 10 seconds and 6 minutes, and more preferably between 40 seconds and 3 minutes, the liquid phase being removed from the reactor, and in which, preferably, the increase in temperature ΔT of the liquid between the inlet and the outlet of the reactor is such that the ratio ΔT/ΔT_{ad} (in which ΔT_{ad} represents the adiabatic temperature increase) is between 0.02 and 0.6 when the ratio between the characteristic heat transfer time tₜₕₑᵣₘ and the characteristic mass transfer time ΔT/ΔT_{ad} is between 1 and 50. This method is used preferably without solvent, i.e. the aromatic compound constitutes the liquid phase that is supplied to the reactor.

### DETAILED DESCRIPTION OF EMBODIMENTS

The invention relates to a continuous method for catalytic reduction (or hydrogenation) of an aromatic compound, preferably a mono- or poly-substituted benzene derivative, into a cycloaliphatic compound. The method is performed in a continuous piston reactor (also called a piston flow reactor), with a length L and a volume V, in which the chemical species (benzene derivative, catalyst and molecular hydrogen) are supplied at one end and move throughout the reactor while being progressively converted. The reactor preferably has a cylindrical shape. It must be equipped with axial agitation means, and preferably axial mechanical agitation means. By axial agitation means, it is meant any device that performs agitation of the reaction mixture over the entire length, or a significant portion thereof, by means having an axis parallel to the axis of the reactor. These axial agitation means facilitate the reaction process, by mixing the chemical species entering with the catalyst, which is in dispersed form in a liquid phase, and facilitates the heat transfer.

The piston reactor has a temperature and concentration profile that may vary along the axis of the reactor. Such a reactor can be modeled as a series of basic reactors arranged in series along an axis and each having a length ΔL and a volume ΔV. In the process conditions of the reactor, the composition of the feed and the total volumetric flow rate F are uniform and constant, and the residence time:

τ=V/F (Equation 1)

The residence time is constant for all of the molecules entering the reactor. This type of reactor is known, and a person skilled in the art also knows that if a highly exothermic reaction is performed in a piston reactor, the radial heat transfer may become limiting. For the hydrogenation of the benzene derivatives, which involves the saturation of at least three conjugated double bonds, the control of heat transfers is, therefore, critical.

The method according to the invention involves a chemical reaction of type:

A(liquid)+νB(gas)−>νₚ Product (Equation 2)

In Equation (2), ν is the stoichiometric coefficient of the gas and νₚ is the stoichiometric coefficient of the product. In accordance with the invention, the gas B is hydrogen, and the benzene derivative to be hydrogenated is in the form of a pure liquid or a liquid diluted in a liquid solvent, or in the form of a solid diluted in a liquid solvent.

In general, the performance of the reactors are given by two characteristic quantities, which respectively describe the heat transfer and the mass transfer. These characteristic transfer times are defined below by simplified equations (the hydrodynamic model is the same, whether the reactor is a piston reactor or a fully agitated reactor, insofar as the hydrogenation reaction is limited by the mass transfer):

the characteristic heat transfer time

$${t}_{therm} = \frac{ρ {C}_{p} {V}_{liq}}{K S} \qquad ( Equation 3 )$$

the characteristic mass transfer time tₘₐₜ=1/(k_{L}a) (Equation 4)

In these equations, the following parameters are used: the density of the liquid ρ; the heat capacity of the liquid Cₚ; the overall transfer coeffᵢcient κ, defined below; the heat eₓchange surface S (constₐnt for a given reactor, becaᵤse it is fixed by the design thereof); the product between the gas-ₗiquid ₘass transfer coefficient on ₜhe liquid side, k_{L}, and the specific interfacial area, a, defined below.

The smaller the characteristic transfer time is, ₜhe more efficient the system is and quickly transfers heat and mass (respectively).

We will now briefly describe the determination of the coefficient K, which is well known to a person skilled in the art.

The overall transfer coefficient K (also called the overall exchange coefficient) is defined by the equation:

Φ=K S ΔTₘₗ (Equation 5)

In Equation 5, S is the exchange surface (in this case S=π D L where D is the internal diameter and L is the internal length of the portion of the tube of the reactor in which the gas comes into contact with the liquid), ΔTₘₗ is the average logarithmic temperature difference:

ΔTₘₗ={[(T(heat transfer fluid)ₒᵤₜₗₑₜ−T(process)]−[(T(heat transfer fluid)ᵢₙₗₑₜ−T(process)ₒᵤₜₗₑₜ]}/ln{[(T(heat transfer fluid)ₒᵤₜₗₑₜ−T(process)ᵢₙₗₑₜ]/[(T(heat transfer fluid)ᵢₙₗₑₜ−T(process)_{outlet]}}

Φ is the power (in Watts, reference temperature 25° C.) obtained by the heat flow on the process side. For a given reaction, these parameters are dependent on the geometry of the reactor and the flow; they can easily be determined.

The coefficient k_{L}a, also well known to a person skilled in the art, can be determined experimentally by a procedure that, so as not to unnecessarily complicate the description of the invention, is described below as “Example 1”.

In an advantageous embodiment of the invention, a continuous piston reactor having the following characteristics is used:
Mass transfer: 0.1 s⁻¹<k_{LA}<0.3 s⁻¹ which is 3 s<tₘₐₜ<10 s
Heat transfer: K=300 to 1000 W/m²/° C. (preferably: 700 W/m²/° C., and even more preferably: around 550 W/m²/° C.)
(in this case, the partial transfer coefficient of the liquid with the metal is considered).

In a typical embodiment, the characteristic transfer time

$${t}_{therm} = \frac{ρ {C}_{p} {V}_{liq}}{K S}$$

is on the order of 25 seconds (with p=1050 kg/m³ Cp=2000 J/kg/° C.).

In this advantageous embodiment, the characteristic time ratio is therefore: 2<(tₜₕₑᵣₘ/tₘₐₜₜ)<8.

In the method according to the invention, the increase in temperature of the liquid ΔT between the inlet and the outlet of the reactor is such that:

$$\frac{Δ T}{Δ {T}_{ad}} = \frac{{t}_{therm}}{( {t}_{therm} + {τ}_{liq} )} {X}_{A} \qquad ( Equation 6 )$$

in which

ΔT_{ad} is the adiabatic temperature increase

$$Δ {T}_{ad} = \frac{( - {Δ}_{r} H ) {C}_{A 0}}{ρ {C}_{p}} \qquad ( Equation 7 )$$

Δ_{Γ}H is the reaction enthalpy,

X_{A} is the stoichiometric coefficient of compound A.

For the case of a total conversion of A (i.e. X_{A}=1), it is possible to rewrite equation (6) as:

$$\frac{Δ T}{( Δ {T}_{ad} )} = \frac{M}{( {t}_{mat} / {t}_{therm} + 1 )} \qquad ( Equation 8 )$$

in which M refers to the stoichiometric ratio:

$$M = \frac{P}{v He {C}_{A 0}} \qquad ( Equation 9 )$$

in which P refers to the working pressure, He refers to the Henry coefficient, and C_{A0} refers to the concentration of liquid at the inlet of the reactor.

The choice of the process conditions of the method according to the invention involves three quantities:

the adiabatic temperature increase in an undiluted medium:

$${( Δ {T}_{ad} )}_{pure} = \frac{( - {Δ}_{r} H ) {( {C}_{A 0} )}_{pure}}{ρ {C}_{p}} \qquad ( Equation 10 )$$

the stoichiometric ratio calculated for the concentration of pure reagents:

$${M}_{pure} = \frac{P}{v {He ( {C}_{A 0} )}_{pure}} \qquad ( Equation 11 )$$

the dilution factor F defined by:

$${( {C}_{A 0} )}_{work} = \frac{{( {C}_{A 0} )}_{pure}}{F} . \qquad ( Equation 12 )$$

The inventors discovered that a particular operating rate of a piston reactor enables the stated problem to be solved. This rate is explained here in the case of a reaction with a stoichiometric coefficient νₚ=1, as is the case for the hydrogenation of the ortho-cresol.

The continuous method can be described as including a plurality of steps. In a first step, a liquid phase that includes the aromatic compound and the dispersed catalyst is continuously supplied, preferably at one end of the reactor. Then, the liquid phase (suspension) is subjected to a temperature of between 100° C. and 300° C. and under axial mechanical agitation, to the influence of a hydrogen pressure of between 10 and 250 bar (preferably between 50 and 250 bar) for a passage time t of between 1 second and 10 minutes (preferably 10 seconds and 6 minutes), and even more preferably 40 seconds to 3 minutes). When the liquid phase has arrived at the other end of the reactor, it is removed from the other end of the reactor, then the catalyst is separated by filtration.

In accordance with the invention, the reaction is performed in such a way that the increase in temperature ΔT of the liquid between the inlet and the outlet of the reactor is such that the ratio ΔT/ΔT_{ad} (in which ΔT_{ad} represents the adiabatic temperature increase) is between 0.02 and 0.6 when the ratio between the characteristic heat transfer time tₜₕₑᵣₘ and the characteristic mass transfer time tₘₐₜ is between 1.5 and 50. This method is used without solvent, i.e., the aromatic compound constitutes the liquid phase supplied to the reactor. In a preferred embodiment, the ratio ΔT/ΔT_{ad} is between 0.02 and 0.2 when tₜₕₑᵣₘ/tₘₐₜ is between 1.5 and 12. In a more preferred embodiment ΔT/ΔT_{ad} is between 0.03 and 0.15 when tₜₕₑᵣₘ/tₘₐₜ is between 2 and 8.

To keep the internal temperature of the reactor constant, the heating power of the reaction chamber is adjusted, given that some of the thermal energy necessary for keeping it at the selected temperature comes from the enthalpy of the hydrogenation reaction taking place in the reaction chamber.

Advantageously, the method in accordance with the invention is implemented in a tubular cylindrical piston reactor with an internal diameter of between 20 mm and 100 mm. Above 100 mm, the productivity of the reactor decreases because, in order for the exchange surface to remain large, the flow rate must be decreased. Below 20 mm, the surface/volume ratio is very large, but the flow rate is insufficient for an industrial-scale production. Preferably, the internal diameter of the piston reactor is between 30 mm and 75 mm, and even more preferably between 40 mm and 60 mm. The length of the reaction chamber of the reactor is between 10 cm and 100 cm. Below 10 cm, the residence time is too short. Above 100 cm, the machining of the tubular reactor becomes difficult, and the agitation of the reaction mixture is difficult to accomplish. A preferred length is between 20 cm and 80 cm.

The reactor must be equipped with axial mechanical agitation means. Different means can be used for this purpose, such as a series of mixers, an endless screw, or an impeller, but these axial mechanical agitation means must not interfere with the “piston” nature of the reactor, as defined by Equation (1).

The method in accordance with the invention implements at least one dispersed catalyst, such as a powder in suspension. Advantageously, this powder includes a substrate (such as alumina, silica or activated carbon) on which a metal element has previously been deposited. As an example, it is possible to use Pd, Ru, Pt or Ni on an activated carbon substrate.

With respect to batch mode methods, the method in accordance with the invention has numerous advantages. Often, the amount of catalyst necessary can be significantly reduced, often by a factor of 5 to 20. For numerous reactions, it is possible to find process conditions in which the reaction yield is greater than or equal to 99%. This high yield makes it possible to do without the additional purification steps that are often necessary in fine chemistry when the yield is less than 95%. It is possible to work in many cases without solvent. The reactor makes it possible to produce industrial amounts of cyclohexane derivatives, for example, on the order of 20 kg/h in the case of ortho-cresol. This makes it possible to achieve an annual production on the order of 80 to 100 tons with a single reactor. In addition, the investment cost of a reactor capable of implementing the method in accordance with the invention is lower than that for a batch-type reactor, and labor requirements are reduced. In addition, the “scaling up” of the method is significantly simplified since the method in accordance with the invention can be implemented in a small continuous industrial reactor, which does not differ significantly from an experimental laboratory reactor. And, finally, the method does not generally use solvent and does not generally produce waste.

The method in accordance with the invention can be applied to numerous chemical compounds. In general, it enables the hydrogenation of an aromatic compound, preferably a mono- or poly-substituted benzene derivative, in a cycloaliphatic compound. More specifically, the method can be applied to mono- or poly-substituted phenol derivatives of formula (I), and it thus produces molecules of formula (II). Examples for the molecules of formula (I) are cresols (ortho-, meta- and para-cresol) and guaiacol.

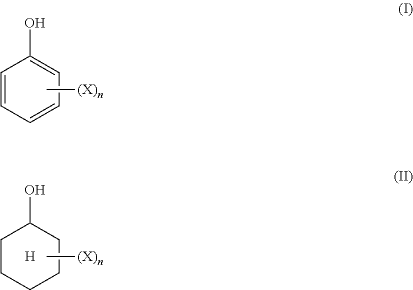

It is also possible to hydrogenate benzene rings with a plurality of hydroxyl groups, such as dihydroxybenzenes (in particular, catechol, resorcinol, hydroquinone) and trihydroxybenzenes (in particular, pyrogallol, phloroglucinol and gallic acid).

Other examples for benzene rings to be hydrogenated are hydroxybenzoic acids (such as salicylic acid) and nitrophenols (such as picric acid).

The method in accordance with the invention can also be applied to molecules of formula (III). In this case, it produces molecules of formula (IV).

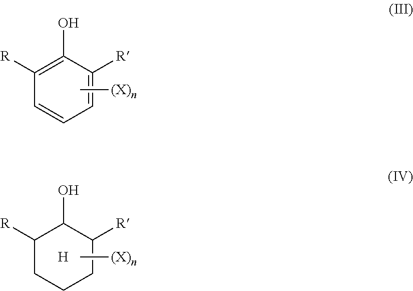

In formulas (I), (II), (III) and (IV), X represents a substituent that is not a hydrogen atom. X can, for example, be an aliphatic group, linear or branched, or an aralkyl group, an alkenyl group, a cycloalkenyl group, a cycloalkyl group or a cycloalkenyl group, in which all of these groups advantageously include between 1 and 36 carbon atoms. As specific examples, the methyl, ethyl, propyl, isopropyl, t-butyl, tridecyl, cyclohexyl, 4-pentadecyloxybenzyl, hexadecyloxycarbonylethyl, 2-ethoxytridecyl, trifluoromethyl and cyclopentyl groups are indicated here.

X can also be an alkoxy group (preferably with 1 to 30 carbon atoms, such as methoxy, ethoxy, 2-methylmethoxy and 2-dodecyloxyethoxy), an alkyl group, a carbamoyloxy group (preferably with 1 to 30 carbon atoms, such as N-ethylcarbamoyloxy and N-phenylcarbamoyloxy), a silyloxy group (preferably with 1 to 30 carbon atoms, such as trimethylsilyloxy and dibutylmethylsilyloxy), an acylamino group (preferably with 2 to 30 carbon atoms such as acetamide, tetradecanamide, 2-(2,4-di-t-amylphenoxy)-acetamide, isopentadecanamide), an alkylamino group (preferably with 1 to 30 carbon atoms, such as methylamino, butylamino, dodecylamino, dimethylamino, diethylamino and methylbutylamino), a ureide group (preferably with 2 to 30 carbon atoms, such as methylureide, phenylureide, N,N-dibutylureide and dimethylureide), an alkenyloxy group (preferably with 2 to 30 carbon atoms, such as 2-propenyloxy), a formyl group, an alkyl-acyl group, an alkyl oxycarbonyl group, an alkyl oxycarbonylamino group or a carbamoyl group (preferably with 1 to 30 carbon atoms such as N-ethylcarbamoyl, N,N-dibutylcarbamoyl, N-(2-dodecyloxyethyl)carbamoyl, N-methyl-N-dodecylcarbamoyl and N-[3-(2,4-di-t-amylphenoxy)propyl]carbamoyl), a phosphonyl group (preferably with 1 to 30 carbon atoms such as phenoxyphosphonyl, octyloxy-phosphonyl and phenylphosphonyl), an imide group (preferably with 1 to 30 carbon atoms such as N-succinimide, hydantoinyl, N-phthalimide and 3-octadecenyl-succin-imide), an azolyl group (such as imidazolyl, pyrazolyl, 3-chloro-pyrazol-1-yl and triazolyl), a halogen atom (such as chlorine and bromine), a hydroxyl group, a cyanide group, a carboxyl group, a nitro group, a linear amine group, and analogous groups.

Preferably, X is a linear or branched alkyl group with 1 to 15 carbon atoms and more preferably with 1 to 8 carbon atoms, an aralkyl group with 7 to 15 carbon atoms and even more preferably with 7 or 8 carbon atoms, or a cycloalkyl group with 3 to 15 carbon atoms and even more preferably with 5 to 8 carbon atoms. In a particularly preferred manner, X is a methyl, ethyl, n-propyl, i-propyl, n-butyl, s-butyl, t-amyl, t-octyl, phenyl or cyclohexyl group. The methyl group is the most preferable of all. The parameter n, an integer, is preferably 0 or 1, and when n is 1, X is preferably located in the para position with respect to the hydroxyl group in formulas (III) and (IV).

In formulas (III) and (IV), R and R′ are independently of one another a tertiary alkyl group. More specifically, R and R′ are represented by formula (V), in which R1, R2 and R3 are, independently of one another, a substituent that replaces the hydrogen radical. These substituents are those indicated for the radical X, insofar as they can be connected to a carbon atom.

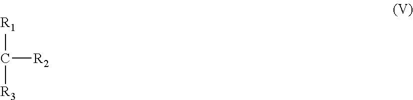

Preferably, R1, R2 and R3 are each a linear or branched alkyl group with 1 to 15 carbon atoms and more preferably with 1 to 8 carbon atoms, an aralkyl group with 7 to 15 carbon atoms and more preferably with 7 or 8 carbon atoms, or a cycloalkyl group with 3 to 15 carbon atoms and even more preferably with 5 to 8 carbon atoms. The linear or branched alkyl groups with 1 to 4 carbon atoms are preferred for R1, R2 and R3 and the methyl group is most preferable of all. R1, R2 and R3 can be identical or different, and can be linked together to form a ring.

Preferred examples for R and W include the t-butyl, t-amyl, t-octyl and 1-methylcyclohexyl groups and the t-butyl group is most preferable for R and R′.

The R and R′ groups located in the two ortho positions with respect to the hydroxyl group of molecules of formula (III) and (IV) can be identical or different, but they are preferably identical.

When R or R′ comprises a substituent capable of being modified during the catalytic hydrogenation, for example if it contains a double bond or an aromatic system, this substituent in formula (IV) can differ from that in formula (III).

In formulas (III) and (IV), the parameter n is an integer between 0 and 3. When n is 2 or 3, X can be the same or different, and a plurality of X can be connected to form a ring. As in the case of R and R′ described above, when X comprises a substituent capable of being modified during catalytic hydrogenation, for example if it contains a double bond or an aromatic system, this substituent in formula (IV) can differ from that in formula (III). In addition, when X becomes, after the catalytic hydrogenation, a hydrogen atom, the parameter n can become a different number. Preferably, n is equal to 1.

In accordance with the invention, preferred compounds represented by formulas (III) and(IV) are those in which R1, R2 and R3 in formula (V) are independently of one another an alkyl group with 1 to 15 carbon atoms, an aralkyl group with 7 to 15 carbon atoms, a cycloalkyl group with 3 to 15 carbon atoms, or an aryl group with 6 to 15 carbon atoms; n is 1, and X is in the para position with respect to the hydroxyl group in formulas (III) and (IV).

More preferable compounds of formulas (III) or (IV) are those in which both R and R′ are t-butyl, t-amyl, t-octyl or 1-methylcyclohexyl groups; X is a methyl, ethyl, n-propyl, iso-propyl, n-butyl, sec-butyl, t-amyl, t-octyl, phenyl or cyclohexyl group; n is 1; and X is located in the para position with respect to the hydroxyl group in formulas (III) and (IV). In particular, the compounds in which X is a methyl group are preferred. More generally, X in this case represents an aliphatic or aryl group, a heterocyclic group, an alkoxy or aryloxy group, a heterocyclic oxy group, an alkylacyloxy or arylacyloxy group, a heterocyclic acyloxy group, a carbamoyloxy, silyloxy, acylamino, alkylamino, arylamino, ureido, alkenyloxy, formyl, alkylacyl or arylacyl group, a heterocyclic acyl group, an alkyloxycarbonyl or aryloxycarbonyl group, a heterocyclic oxycarbonyl group, an alkyloxycarbonylamino or aryoxycarbonylamino group, a heterocyclic oxycarbonylamino group, a carbamoyl, phosphonyl, imide or azolyl group, a halogen atom, a hydroxyl, cyano, carboxy or nitro group, or an unsubstituted amino group.

The invention is illustrated below in Examples 1 to 3, which do not, however, limit the scope of the invention. Example 1 relates to a chemical reaction that falls within the scope of the present invention, but it serves in particular here to describe an experimental procedure that can be used to determine the k_{L}a parameter of a piston reactor.

### EXAMPLE 1

Here is presented an experimental method that can be used to determine the k_{L}a parameter of a reactor. This method also illustrates an example of an embodiment of the invention.

The product k_{L}a for a given reactor is determined from a well known chemical reaction, namely the catalytic hydrogenation of nitrobenzene into aniline (Ph-NO₂+3H2→Ph-NH₂+2H₂O, in which Ph refers to a phenyl group). This reaction is performed in the liquid phase without solvent, and the gas phase consists of pure hydrogen at an initial pressure of 2 bars. The catalyst consists of carbon powder (particle diameter equivalent to approximately 50 μm) filled with 5% palladium mass. The mass concentration of the catalyst is 2.5 g/l and the hydrogenation is performed at ambient temperature. A quartz pressure sensor makes it possible to measure the hydrogen pressure as a function of time. The reactor has a double casing; circulation of thermostatically controlled water inside the double casing makes it possible to keep the temperature of the reactor constant.

At the start, the non-agitated reactor is kept under nitrogen pressure; it is then purged with hydrogen. At a hydrogen pressure of 2 bars, the agitation is started, and the reduction in hydrogen pressure is recorded. The reaction is allowed to continue until the pressure reaches a value of 0.5 atm. Then, the agitation stops and the apparatus is re-pressurized with hydrogen; then after around ten minutes, the measurement is repeated with a different agitation rate. For each test performed, it is noted that the hydrogen pressure decreases exponentially.

Thus, by plotting ln P_{H2}/P₀=f(t), a straight line is obtained in which the slope makes it possible to determine the product α kₐₚₚ. If the change in this product α kₐₚₚ with the agitation rate is plotted, asymptotic behavior is observed. For low agitation rates, the apparent conductance increases with the agitation rate; this indicates a limitation in the apparent kinetics for the gas-liquid transfer. For high agitation rates, a plateau is reached; this means that the transfer is limited, either by the chemical kinetics or by the kinetics of the liquid-solid mass transfer. The development of the curve α kₐₚₚ =f(agitation rate) then makes it possible to estimate the transfer conductance value k_{L}a. In practice, five to six experimental points are sufficient to determine this value.

Here, it is presented the theoretical bases for this determination of k_{L}a. By disregarding the accumulation of liquid phase hydrogen, the expression of tₕe hydrogen dissipation flux in a closed reactor can be established.

$$\frac{d {n}_{{H}_{2}}}{d t} = - \frac{{V}_{G}}{RT} \frac{d {P}_{{H}_{2}}}{d t} = {ϕ}_{{H}_{2}} {V}_{R} \qquad ( 1 )$$

Here, Φ_{H2} is the specific dissipation flux of hydrogen. This flow can be expressed by showing the reaction rate or the transfer flow:

$${ϕ}_{{H}_{2}} = {r}_{v} α = {( {K}_{{H}_{2}} a )}_{global} ( \frac{{P}_{{H}_{2}}}{He} - {C}_{{H}_{2}}^{surface} ) \qquad ( 2 )$$

Here, r_{ν} is the volumetric reaction rate of the hydrogenation, α is the solid retention in the reactor and (K_{H2}a)_{global} is the overall transfer conductance of the hydrogen from the gas phase to the surface of the catalyst.

As the volume of the catalyst and the nitrobenzene concentration are considered to be invariant during a test, the reaction rate can be expressed as resulting from first-order kinetics with respect to the hydrogen concentration, that is:

$${r}_{v} = η {k}_{v} {C}_{NB} {C}_{{H}_{2}}^{surface} = {k}_{v}^{′} {C}_{{H}_{2}}^{surface} \qquad ( 3 )$$

When the gas phase is pure hydrogen, the overall transfer conductance can be expressed as a function of the partial gas-liquid and liquid-solid transfer conductances by:

$$\frac{1}{{[ {K}_{{H}_{2}} a ]}_{global}} = \frac{1}{{k}_{L} {a}_{LG}} + \frac{1}{{k}_{S} {a}_{S} α} \qquad ( 4 )$$

Here, aₛ is the specific surface of the solid and a_{w} is the specific surface of the gas-liquid. By combining the expressions of the chemical kinetics and the physical kinetics, the specific hydrogen dissipation flux in the reactor can be expressed by:

$${ϕ}_{{H}_{2}} = α {k}_{app} \frac{{P}_{{H}_{2}}}{He} \qquad ( 5 )$$

Here, α kₐₚₚ is an apparent conductance that integrates the limitations due to the chemical kinetics, but also the limitations due to the physical kinetics.

$$\frac{1}{α {k}_{app}} = \frac{1}{{[ {K}_{{H}_{2}} a ]}_{global}} + \frac{1}{α {k}_{v}^{′}} = \frac{1}{{k}_{L} {a}_{LG}} + \frac{1}{α} [ \frac{1}{{k}_{s} {a}_{s}} + \frac{1}{{k}_{v}^{′}} ] \qquad ( 6 )$$

By injecting (5) into (1), the following is obtained:

$$\frac{{dP}_{{H}_{2}}}{{P}_{{H}_{2}}} = - α {k}_{app} \frac{{V}_{R}}{{V}_{G}} \frac{RT}{He} dt \qquad ( 7 )$$

of which the integration leads to:

$$ln \frac{{P}_{{H}_{2}}}{{P}_{0}} = - α {k}_{app} \frac{{V}_{R}}{{V}_{G}} \frac{RT}{He} ( t - {t}_{0} ) \qquad ( 8 )$$

The interpretation of the change in hydrogen pressure in a closed system thus makes it possible to determine the apparent conductance of the system. This makes it possible to obtain the gas-liquid transfer conductance value.

### EXAMPLE 2

The use of the method in accordance with the invention for catalytic hydrogenation of ortho-cresol has already been described above in detail. This reaction was performed without solvent. A suspension comprised of molten ortho-cresol and 0.4% by weight of a nickel-type catalyst on a carbon substrate is introduced into the piston reactor by means of a pump. The mixture is continuously pre-heated outside the reactor at a temperature of around 180° C. The hydrogen is kept at a pressure of around 150 bar. For a residence time of around 2 minutes and thirty seconds, the conversion under these conditions is greater than 99.9%. At the outlet of the reactor (outlet temperature of around 280° C.), after depressurization and filtration of the catalyst, the methylcyclohexanol is obtained with a purity greater than 99.0% as verified by gas phase chromatography.

### EXAMPLE 3

Here, for the specific case of hydrogenation of ortho-cresol, the productivity of the method in accordance with the invention is compared with a continuous method in accordance with the prior art.

For the batch reactor, a 6 m³ agitated hydrogenation tank, filled around two-thirds (i.e. around 4000 liters) is used. A reaction mixture consisting of a volume or ortho-cresol and 5 volumes of ethanol as the solvent, i.e. around 670 kg of ortho-cresol for around 3350 liters of ethanol is used. The hydrogenation reaction is conducted at 100° C. under a hydrogen pressure of less than 10 bar for 4 hours, in the presence of a catalyst, until complete conversion. The time of the full cycle is around 30 hours, including the filling of the reactor, the inerting (vacuumizing, rinsing with nitrogen), the hydrogenating (p<10 bar), the hydrogenation reaction itself, the cooling, the filtration of the catalyst and the distillation of the solvent. For 670 kg of added ortho-cresol, 672 kg of methylcyclohexanol and 34 kg of non-reacted ortho-cresol are obtained. This corresponds to a yield of 95% and a productivity of 22.4 kg/h.

With the method in accordance with the invention, conducted without solvent and in the presence of a dispersed catalyst, in a continuous reactor of which the liquid reaction volume is V_{liq}=0.7 liters, a productivity (at 150 bar with a mass transfer time tₘₐₜ=3.5 s) on the order of 5.5×10⁻² mol/s, i.e. a cyclohexanol mass flow rate (M=114 g/mol) on the order of 22 kg/h is obtained.

For a continuous reactor, the productivity of the method in accordance with the invention as determined above is entirely beneficial on an industrial level, even in the case of a simple molecule such as ortho-cresol; it is even more so for more complicated molecules. To increase productivity, it is possible to increase the diameter of the reactor, but this possibility is limited by the heat transfer, as explained above. Advantageously, a plurality of reactors is used, given their simplicity, the continuous nature of the method and the fact that this continuous method does not require much labor.

1-10. (canceled)

11. A method to catalytically hydrogenate an aromatic compound into a cycloaliphatic compound comprising:
(a) continuously supplying at least one liquid phase comprising said aromatic compound and a catalyst dispersed in said liquid phase to a piston reactor;
(b) subjecting said at least one liquid phase to an influence of a hydrogen pressure of between approximately 10 bar and 250 bar at a temperature of between approximately 100° C. and 300° C., under axial agitation, in the presence of said catalyst dispersed in said liquid phase, and for a passage time (t) of between approximately 10 seconds and 10 minutes; and then
(c) removing said liquid phase from said reactor,
wherein the increase in temperature (ΔT) of said liquid phase between an inlet and an outlet of said piston reactor is such that the ratio between the increase in temperature (ΔT) and the increase in adiabatic temperature (ΔT_{ad}) is between approximately 0.02 and 0.6 when the ratio between the characteristic heat transfer time (tₜₕₑᵣₘ) and the characteristic mass transfer time (tₘₐₜ) is between approximately 1 and 50.

12. The method of claim 11, wherein said ratio ΔT/ΔT_{ad} is between approximately 0.02 and 0.2 when said ratio tₜₕₑᵣₘ/tₘₐₜ is between approximately 1.5 and 12 or is between approximately 0.03 and 0.15 when said ratio tₜₕₑᵣₘ/tₘₐₜ is between approximately 2 and 8.

13. The method of claim 11, wherein tₘₐₜ is between approximately 3 seconds and 10 seconds.

14. The method of claim 11, further comprising a heat transfer value between approximately 300 W/m²/° C. and 700 W/m²/° C.

15. The method of claim 11, wherein said aromatic compound constitutes said liquid phase supplied to said piston reactor.

16. The method of claim 11, wherein said aromatic compound comprises a mono-substituted derivative of benzene or a poly-substituted derivative of benzene.

17. The method of claim 11, wherein said piston reactor comprises the approximate shape of a cylinder, said axial agitation comprises axial mechanical agitation, and said at least one liquid phase is continuously supplied at one end of said piston reactor.

18. The method of claim 11, wherein said hydrogen pressure is between approximately 50 bar and 250 bar.

19. The method of claim 11, wherein said passage of time is one of:
between approximately 10 seconds and 6 minutes; and
between approximately 40 seconds and 3 minutes.

20. The method of claim 11, wherein said aromatic compound comprises at least one of a mono-substituted phenol, a poly-substituted phenol, an unsubstituted polyhydroxybenzene, a mono-polyhydroxybenzene, and a poly-substituted polyhydroxybenzene.

21. The method of claim 20, wherein said aromatic compound is represented by formula (I) and said cycloaliphatic compound is as represented by formula (II), 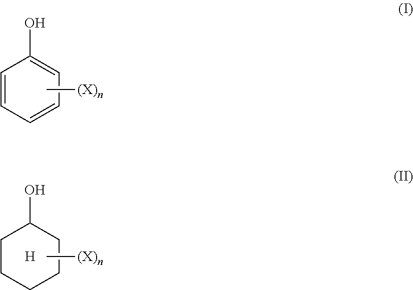
wherein X is a substituent that is not a hydrogen atom and n is an integer between 0 and 3.

22. The method of claim 21, wherein X comprises:
(a) a linear or branched aliphatic group, an aralkyl group, an alkenyl group, a cycloalkenyl group, a cycloalkyl group or a cycloalkenyl group;
(b) an alkoxy group, an alkyl group, a carbamoyloxy group, a silyloxy group, an acylamino group, an alkylamino group, a ureide group, an alkenyloxy group, a formyl group, an alkyl-acyl group, an alkyl oxycarbonyl group, an alkyl oxycarbonylamino group, a carbamoyl group, a phosphonyl group, an imide group, an azolyl group, a halogen atom, a hydroxyl group, a cyanide group, a carboxyl group, a nitro group, or a linear amine group;
(c) a linear or branched alkyl group, an aralkyl group, or a cycloalkyl group; or
(d) a methyl, ethyl, n-propyl, i-propyl, n-butyl, s-butyl, t-amyl, t-octyl, phenyl or cyclohexyl group.

23. The method of claim 22, wherein said aliphatic group, aralkyl group, alkenyl group, cycloalkenyl group, cycloalkyl group or cycloalkenyl group includes between 1 and 36 carbon atoms.

24. The method of claim 23, further comprising a methyl, ethyl, propyl, isopropyl, t-butyl, tridecyl, cyclohexyl, 4-pentadecyloxybenzyl, hexadecyloxycarbonylethyl, 2-ethoxytridecyl, trifluoromethyl or cyclopentyl group.

25. The method of claim 22, wherein said alkoxy group includes 1 to 30 carbon atoms, said alkyl group includes 1 to 30 carbon atoms, said silyloxy group includes 1 to 30 carbon atoms, said acylamino group includes 2 to 30 carbon atoms, said alkylamino group includes 1 to 30 carbon atoms, said ureide group includes 2 to 30 carbon atoms, said alkenyloxy group includes 2 to 30 carbon atoms, said alkyl oxycarbonylamino group or carbamoyl group includes 1 to 30 carbon atoms, said phosphonyl group includes 1 to 30 carbon atoms, and said imide group includes 1 to 30 carbon atoms.

26. The method of claim 25, wherein said alkoxy group includes methoxy, ethoxy, 2-methylmethoxy and 2-dodecyloxyethoxy, said carbamoyloxy group includes N-ethylcarbamoyloxy and N-phenylcarbamoyloxy, said silyloxy group includes trimethylsilyloxy and dibutylmethylsilyloxy, said acylamino includes acetamide, tetradecanamide, 2-(2,4-di-t-amylphenoxy)-acetamide, and isopentadecanamide, said alkylamino group includes methylamino, butylamino, dodecylamino, dimethylamino, diethylamino and methylbutylamino, said ureide group includes methylureide, phenylureide, N,N-dibutylureide and dimethylureide, said alkenyloxy group includes 2-propenyloxy, said carbamoyl group includes N-ethylcarbamoyl, N,N-dibutylcarbamoyl, N-(2-dodecyloxyethyl)carbamoyl, N-methyl-N-dodecylcarbamoyl and N-[3-(2,4-di-t-amylphenoxy)propyl]carbamoyl, said phosphonyl group includes phenoxyphosphonyl, octyloxy-phosphonyl and phenylphosphonyl, said imide group includes N-succinimide, hydantoinyl, N-phthalimide and 3-octadecenyl-succin-imide, said azolyl group includes imidazolyl, pyrazolyl, 3-chloro-pyrazol-1-yl and triazolyl, and said halogen atom includes chlorine and bromine.

27. The method of claim 22, wherein said linear or branched alkyl group includes 1 to 15 carbon atoms, said aralkyl group includes 7 to 15 carbon atoms, and said cycloalkyl group includes 3 to 15 carbon atoms.

28. The method of claim 27, wherein said linear or branched alkyl group includes 1 to 8 carbon atoms, said aralkyl group includes 7 or 8 carbon atoms, or said cycloalkyl group includes 5 to 8 carbon atoms.

29. The method of claim 20, wherein said aromatic compound is represented by formula (Ill) and said cycloaliphatic compound is as represented by formula (IV), 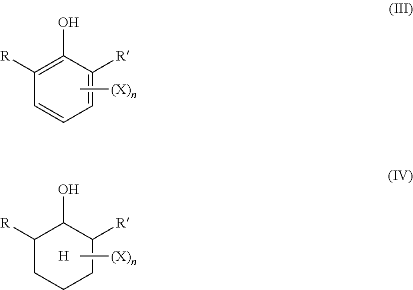
wherein R and R′ are, independently of one another, a tertiary alkyl group, X is a substituent that is not a hydrogen atom, and n is an integer between 0 and 3.

30. The method of claim 29, wherein X comprises:
(a) a linear or branched aliphatic group, an aralkyl group, an alkenyl group, a cycloalkenyl group, a cycloalkyl group or a cycloalkenyl group;
(b) an alkoxy group, an alkyl group, a carbamoyloxy group, a silyloxy group, an acylamino group, an alkylamino group, a ureide group, an alkenyloxy group, a formyl group, an alkyl-acyl group, an alkyl oxycarbonyl group, an alkyl oxycarbonylamino group, a carbamoyl group, a phosphonyl group, an imide group, an azolyl group, a halogen atom, a hydroxyl group, a cyanide group, a carboxyl group, a nitro group, or a linear amine group;
(c) a linear or branched alkyl group, an aralkyl group, or a cycloalkyl group; or
(d) a methyl, ethyl, n-propyl, i-propyl, n-butyl, s-butyl, t-amyl, t-octyl, phenyl or cyclohexyl group.
